(19) United States
(12) Patent Application Publication
(10) Pub. No.: US 2015/0148383 A1
(43) Pub. Date: May 28, 2015

(54) Methods for Inhibiting or Reversing Epiretinal Membrane Formation

(71) Applicant: Regenerative Research Foundation, Rensselaer, NY (US)

(72) Inventors: Jeffrey Stern, Slingerlands, NY (US); Sally Temple Stern, Slingerlands, NY (US); Timothy Blenkinsop, Rensselaer, NY (US)

(21) Appl. No.: 14/550,304

(22) Filed: Nov. 21, 2014

Related U.S. Application Data
(60) Provisional application No. 61/907,979, filed on Nov. 22, 2013.

(51) Int. Cl. A61K 31/455

(52) U.S. Cl. CPC A61K 31/455
USPC 514/355

(57) ABSTRACT

Described herein are treatment methods involving the administration of nicotinamide, which has been discovered to stabilize the normal phenotype of retinal pigment epithelial cells and to prevent retinal pigment epithelial cell proliferation. Diseases and disorders that can be treated according to the methods disclosed herein include, e.g., macular pucker, proliferative vitreoretinopathy, preretinal fibrosis, vitreomacular traction, tractional retinal detachment, and phthsis bulbi, cystoid macular edema (CME) arising from intraocular inflammation due to uveitis, vasculitis, trauma, surgery, and collagen vascular disease (e.g., Behcets disease or sarcoidosis), and age related macular degeneration.

### TECHNICAL FIELD

This invention relates to generally to the field of cellular and molecular biology.

### BACKGROUND

Nicotinamide is a water soluble B vitamin also known as niacinamide and nicotinic acid amide. In cells, niacin is incorporated into nicotinamide adenine dinucleotide (NAD) and nicotinamide adenine dinucleotide phosphate (NADP), although the pathways for nicotinamide and nicotinic acid are very similar. NAD+ and NADP+ are coenzymes in a wide variety of enzymatic oxidation-reduction reactions. Nicotinamide has a wide range of beneficial effects and is highly safe for human use. Nicotinamide promotes the differentiation of RPE stem cells into RPE cell progeny [see, Blenkinsop et al. Methods Mol Biol. 2013; 945:45-65].

The retinal pigment epithelium (RPE) is a monolayer of pigmented cells located between the neural retinal photoreceptors and the Bruch's basement membrane shared by the RPE and choroid. Normal adult RPE is a stable, terminally differentiated tissue that does not proliferate. In disease, however, the RPE cells can undergo phenotypic changes into abnormal cell types associated with disease states. In some diseases, these pathologic RPE phenotypes proliferate to advance the disease. In other disease states, dysfunctional RPE cells produce non-proliferative pathologic effects that mediate disease progression. Pathologic RPE phenotypes and/or dysfunctional RPE cells are a hallmark of a number of disease states of the eye, including, e.g., macular pucker, proliferative vitreoretinopathy (PVR), preretinal fibrosis, and age-related macular degeneration (AMD). See, e.g., Marmor M F, Wolfensberger T J (eds): The Retinal Pigment Epithelium: Current Aspects of Function and Disease. Oxford University Press, New York (1998).

Currently, effective treatments for these and other RPE-associated disease states are lacking and/or have undesirable side effects. Thus, novel therapies that stabilize RPE phenotypes and/or that can inhibit the phenotypic change and/or cell death which can occur in AMD and other retinal diseases are needed.

### SUMMARY

As follows from the Background section, above, there is a need in the art for novel therapies that stabilize RPE phenotypes and that can inhibit the phenotypic change and/or cell death that occurs in AMD and other retinal diseases. These and other, related advantages, are provided herein.

In one aspect, a method for inhibiting or reversing epiretinal membrane (ERM) formation in a patient is provided. The method can include administering to a patient suffering from or at risk of developing ERM an amount of nicotinamide effective for inhibiting proliferation of retinal pigmented epithelial (RPE) cells. In some aspects of the method, ERM is the underlying disease state of a condition such as, e.g., macular pucker, proliferative vitreoretinopathy, preretinal fibrosis, vitreomacular traction, tractional retinal detachment, or phthsis bulbi.

In another aspect, a method for inhibiting or reversing epithelial mesenchymal transition (EMT) in a patient is provided. The method can include administering to a patient suffering from or at risk of developing EMT an amount of nicotinamide effective for maintaining or inducing normal RPE cell morphology.

In another aspect, a method for treating a retinal disease or condition associated with cystoid macular edema (CME) in a patient is provided. The method can include administering to a patient in need thereof an amount of nicotinamide effective for reducing leakage through tight junctions that causes CME. In some aspects of the method, the retinal disease or condition causing CME can be, e.g., intraocular inflammation due to uveitis, vasculitis, trauma, surgery, or collagen vascular disease. In some aspects of the method, the collagen vascular disease is Behcets disease or sarcoidosis.

In another aspect, a method for inhibiting a pathologic condition associated with oxidative damage-induced RPE atrophy in a patient is provided. The method can include administering to a patient in need thereof an amount of nicotinamide effective for inhibiting stress-induced RPE drusen formation and/or stress-induced RPE cell death and/or RPE cell atrophy. In some aspects of the method, the pathologic condition is dry (non-exudative) AMD.

In another aspect, a method for inhibiting drusen in a patient is provided. The method can include administering to a patient in need thereof an amount of nicotinamide effective for stabilizing the RPE phenotype, thereby preventing overproduction of drusen protein and associated drusen. In some aspects, the patient is suffering from dry (non-exudative) AMD.

In another aspect, a method for treating a patient suffering from or at risk of developing neovascular (wet or exudative) AMD is provided. The method can include administering to a patient in need thereof an amount of nicotinamide effective for stabilizing the RPE cell barrier to choroidal neovascular ingrowth.

In any of the above aspects, the patient can be a mammal (e.g., a human).

Unless otherwise defined, all technical and scientific terms used herein have the same meaning as commonly understood by one of ordinary skill in the art to which this disclosure pertains. In case of conflict, the present document, including definitions, will control.

All publications, patent applications, patents, and other references mentioned herein are incorporated by reference in their entireties. The materials, methods, and examples disclosed herein are illustrative only and not intended to be limiting.

The details of one or more embodiments of the disclosure are set forth in the accompanying drawings and the description below. Preferred methods and materials are described below, although methods and materials similar or equivalent to those described herein can also be used in the practice or testing of the present disclosure. Other features, objects, and advantages of the present disclosure will be apparent from the description and drawings, and from the claims.

### DESCRIPTION OF DRAWINGS

FIGS. IA-1B are each a photograph of a light microscopic image of RPE cells cultured in the absence (FIG. 1A) or presence (FIG. 1B) of 10 mM nicotinamide; scale bar=50 μm.

FIG. 2 is a line graph plotting resistance on the Y-axis versus time on the X-axis as measured in RPE cells treated in the presence (+) or absence (−) of nicotinamide (“Nic”); ** indicates P value <0.01.

FIGS. 3A-3B are each a photograph of a fluorescence microscopic image of RPE cells cultured in the absence (FIG. 3A) or presence (FIG. 3B) of 10 mM nicotinamide and stained with DAPI (nuclear stain) and rabbit anti-ZO-1 specific antibody (Invitrogen) with Alexafluor 488 secondary antibody (Invitrogen). The lighter gray regions indicate positive staining for ZO-1; scale bar=10 μm.

FIG. 4 contains bar graphs quantifying the relative mRNA expression (relative to control) of MITF and RPE65 in RPE cells treated with 10 mM nicotinamide (“Nic”); ** indicates statistical significance with P value <0.01 using the paired t-test.

FIG. 5 contains bar graphs quantifying the relative mRNA expression (relative to control) of SNAIL, SLUG and TWIST in RPE cells treated with 10 mM nicotinamide (“Nic”); ** indicates statistical significance with P value <0.01; * indicates statistical significance with P value <0.05 using the paired t-test.

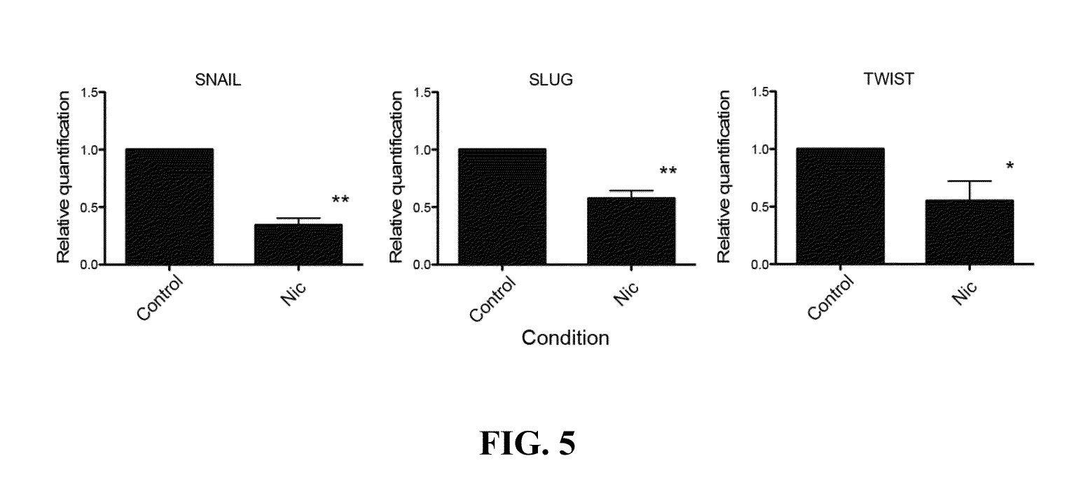

FIGS. 6A-6B are each a photograph of a microscopic image of fluorescently-stained RPE cells following culture in the absence (FIG. 6A) or presence (FIG. 6B) of 10 mm nicotinamide. Cells were stained with DAPI (nuclear stain) and a Ki67-specific fluorescently-labeled antibody; scale bar=50 μm.

FIG. 7 contains bar graphs quantifying the mRNA expression level (relative to Day 0) of TIMP-3 (a marker of induction of oxidative stress) in RPE cells cultured in the absence (left graph) or presence (right graph) of nicotinamide.

FIGS. 8A-8D contain representative photographs of images collected using a fundus camera.

FIGS. 9A-9D contain representative photographs of images collected using a fundus camera (column 1) (same images as in FIGS. 8A-8B), Optko or Heidleberg optical coherence tomography (OCT) (column 2), and fluorescein angiogram results (column 3) in patients that received antibody to vascular endothelial growth factor (VEGF) (e.g., avastin, lucentis or eylea).

FIG. 10 contains photographs of microscopic images (magnification 32×) of fibroblastic cell cultures at the time just prior to addition of 10 mM nicotinamide (initial culture), and at 3 and 8 weeks after continuous culture in the presence of 10 mM nicotinamide.

### DETAILED DESCRIPTION

### Overview

The retinal pigment epithelium (RPE) layer of the retina has essential roles in supporting the function of overlying neural retinal tissue. Loss of the RPE cell phenotype, e.g., through atrophy or change of cell type, as well as uncontrolled proliferation of RPE cells, results in retinal dysfunction and disease. The present disclose provides novel treatment methods involving the administration of nicotinamide or a related agent to patients in need thereof. In particular, while not intending to be bound by any particular theory or mechanism of action, it is presently discovered that nicotinamide stabilizes RPE cells and protects against RPE atrophy and/or change in phenotype, e.g. by maintaining and/or enhancing RPE morphology, by inhibiting RPE cell proliferation, and/or by inhibiting RPE cells from undergoing epithelial to mesenchymal transition (EMT), which can lead to epiretinal membrane (“ERM”) formation and development of retinal traction.

Thus, presently disclosed are methods for inhibiting or reversing ERM in a patient. The methods can include administering nicotinamide to a patient suffering from or at risk of developing ERM. e.g., in an amount effective for inhibiting proliferation of RPE cells. Also disclosed are methods for inhibiting epithelial to mesenchymal transition (EMT). The methods can include administering nicotinamide to a patient suffering from or at risk of developing EMT, e.g., in an amount effective for stabilizing RPE phenotype. ERM is an underlying disease state associated with numerous conditions of the retina, including, but not limited to: macular pucker, proliferative vitreoretinopathy, preretinal fibrosis, vitreomacular traction, tractional retinal detachment, and phthsis bulbi. Thus, the foregoing conditions are non-limiting examples of conditions of the retina that can be treated according to the presently disclosed methods.

It is also discovered that nicotinamide reduces leakage through vascular tight junctions in RPE cells. Thus, disclosed herein are methods for treating retinal diseases or conditions associated with CME, e.g., intraocular inflammation due to uveitis, vasculitis, trauma, surgery, and collagen vascular disease. The methods can include administering nicotinamide to a patient in need thereof, e.g., in an amount effective for reducing leakage through RPE cell vascular tight junctions.

It is also discovered that nicotinamide blocks certain responses to oxidative stress in RPE cells, which can lead to cell damage and death, and occurs in conditions such as age related macular degeneration (AMD). Furthermore, oxidative stress induces abnormal RPE phenotypes that are responsible for the production of drusen protein, which is an underlying cause of dry AMD. Administration of nicotinamide to AMD patients can reduce or inhibit the abnormal RPE phenotypes associated with drusen overexpression, thereby leading to inhibition of drusen overexpression. Thus, also provided herein are methods for treating AMD by administering nicotinamide to a patient suffering from or at risk of developing AMD (e.g., dry AMD), e.g., in an amount effective for inhibiting drusen expression. Also provided herein are methods for treating wet AMD.

As discussed in more detail below, nicotinamide can also be used to stabilize transplanted RPE cells, as well as to stabilize RPE cells that have been activated by pharmacologic manipulation or other treatments/traumas (e.g., eye injury or surgery).

### DEFINITIONS

As used herein, “epiretinal membrane” or “ERM” is a condition characterized by the proliferation of RPE cells, their migration over the inner retina and into the vitreous (often via defects in the retina caused by retinal tears or surgical manipulations). As used herein, the phrase “inhibiting or reversing epiretinal membrane (ERM)” means reducing or eliminating the proliferation of RPE cells, their transformation into mesenchymal progeny (e.g., fibroblasts, myofibroblasts, osteoblasts, etc.), their migration into the retina and/or vitreous, and their distortion of the retinal architecture.

As used herein, the phrase “inhibiting epithelial mesenchymal transition (EMT)” means preventing the conversion of RPE phenotype into mesenchyme phenotypes. As used herein, the phrase “reversing EMT” means causing mesenchymal phenotypes derived from RPE cells to revert to the RPE phenotype.

As used herein, “normal RPE cell morphology” means the typical cobblestone hexagonal morphology characteristic of RPE cells. Normal RPE cell morphology is part of the “RPE phenotype,” which also includes markers, physiology and other characteristic morphologies which are known in the art [see, e.g., Blenkinsop et al. Methods Mol Biol. 2013; 945:45-65]. As used herein. “maintaining normal RPE cell morphology” means inhibiting RPE cells that already have the characteristic cobblestone morphology from losing their cobblestone morphology. As used herein, “inducing normal RPE cell morphology” means causing RPE cells that have lost normal RPE cell morphology to acquire the typical cobblestone morphology of RPE cells.

As used herein, a patient who is “suffering from” a particular disease, condition, or disorder (e.g., a disease, condition or disorder affecting the retina), exhibits one or more symptoms or signs of the disease, condition or disorder.

As used herein, a patient who is “at risk of developing” a particular disease, condition, or disorder (e.g., a disease, condition or disorder affecting the retina) exhibits one or more warning signs or symptoms of developing the particular disease, condition or disorder. Such warning signs or symptoms can be, e.g., loss or distortion of vision, or the appearance of abnormal cellular material on or within the retina detected ophthalmoscopically or by OCT (optical coherence tomography), leakage of fluorescein dye detected using fluorescein angiography, macular edema, retinal vein occlusion, diabetic retinopathy, retinal vasculitis, retinal hemorrhage, a retinal break, ocular trauma, the presence of vitreous cells, anterior chamber cells, or haziness (flare) in the anterior chamber or vitreous, cells with an abnormal phenotype located on, within or under the retina, or abnormal differentiation of RPE cells evidenced by pigment hypertrophy, pigment hypotrophy, or an excess accumulation of cells, on, within or under the retina, and/or any other sign of epithelial to mesenchymal transition, or “EMT”, resulting in abnormal proliferation and/or abnormal phenotype of RPE cells.

As used herein, “inhibition of RPE proliferation” means any reduction in the amount of RPE proliferation, e.g., relative to the amount of proliferation prior to treatment. Thus, for example, inhibition of RPE proliferation includes e.g., a reduction in proliferation of at least about, e.g., 5%, 10%, 15%, 20%, 35%, 30%, 35%, 40%, 45%, 50%, 55%, 60%, 65%, 70%, 75%, 80%, 85%, 90%, 95%, 96%, 97%, 98%, 99%, or 100%, relative to the amount of proliferation occurring or expected to occur prior to treatment e.g., treatment with nicotinamide or a nicotinamide-related compound).

As used herein, “reducing leakage through vascular tight junctions” means any reduction in vascular tight junction leakage, e.g., relative to the amount of leakage prior to treatment. Thus, for example, the methods disclosed herein encompass reductions in vascular tight junction leakage of at least about, e.g., 5%, 10%, 15%, 20%, 35%, 30%, 35%, 40%, 45%, 50%, 55%, 60%, 65%, 70%, 75%, 80%, 85%, 90%, 95%, 96%, 97%, 98%, 99%, or 100%, relative to the amount of leakage prior to treatment (e.g., treatment with nicotinamide or a nicotinamide-related compound).

As used herein, “inhibiting drusen expression” means any reduction in drusen or rate of drusen formation, e.g., relative to the amount of drusen or rate of drusen formation prior to treatment. Thus, for example, the methods disclosed herein encompass reductions in drusen expression of at least about, e.g., 5%, 10%, 15%, 20%, 35%, 30%, 35%, 40%, 45%, 50%, 55%, 60%, 65%, 70%, 75%, 80%, 85%, 90%, 95%, 96%, 97%, 98%, 99%, or 100%, relative to the amount of drusen present or expected to form prior to treatment (e.g., treatment with nicotinamide or a nicotinamide-related compound).

As used herein, the term “about” or “approximately” means within an acceptable error range for the particular value as determined by one of ordinary skill in the art, is which will depend in part on how the value is measured or determined, i.e., the limitations of the measurement system. For example, “about” can mean within 1 or more than I standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

The terms “therapeutically effective” and “effective amount,” used interchangeable, refer to that quantity of a composition, compound or pharmaceutical formulation that is sufficient to reduce or eliminate at least one symptom of a disease or condition specified herein, e.g., cancer. When a combination of active ingredients is administered, the effective amount of the combination may or may not include amounts of each ingredient that would have been effective if administered individually. The dosage of the therapeutic formulation will vary, depending upon the nature of the disease or condition, the patient's medical history, the frequency of administration, the manner of administration, the clearance of the agent from the host, and the like. The initial dose may be larger, followed by smaller maintenance doses. The dose may be administered. e.g., as needed, monthly, weekly, daily, etc., to maintain an effective dosage level. Therapeutically effective dosages can be determined stepwise by combinations of approaches such as (i) characterization of effective doses of the composition or compound in in vitro cell culture assays using cell phenotype, growth and/or survival as a readout followed by (ii) characterization in animal studies using cell phenotype, growth and/or survival as a readout, followed by (iii) characterization in human trials using cell growth, phenotype and/or survival as a readout.

As used herein, the term “subject” means any mammal, and, in particular, a human, and can also be referred to, e.g., as an individual or patient. A non-human mammal can be, for example, and without limitation, a non-human primate (such as a monkey, baboon, gorilla, or orangutan), a bovine animal, a horse, a whale, a dolphin, a sheep, a goat, a pig, a dog, a feline animal (such as a cat), a rabbit, a guinea pig, a hamster, a gerbil, a rat, or a mouse.

As used herein, “treating” or “treatment” of a state, disorder or condition includes: (1) preventing or delaying the appearance of clinical or sub-clinical signs or symptoms of the state, disorder or condition developing in a mammal that may be afflicted with or predisposed to the state, disorder or condition but does not yet experience or display clinical or subclinical signs or symptoms of the state, disorder or condition; and/or (2) inhibiting the state, disorder or condition, i.e., arresting, reducing or delaying the development of the disease or a relapse thereof (in case of maintenance treatment) or at least one clinical or sub-clinical sign or symptom thereof; and/or (3) relieving the disease, i.e., causing regression of the state, disorder or condition or at least one of its clinical or sub-clinical signs or symptoms; and/or (4) causing a decrease in the severity of one or more signs or symptoms of the disease. The benefit to a subject to be treated is either statistically significant or at least perceptible to the patient or to the physician.

The methods disclosed herein involve administration of nicotinamide or a related compound for, e.g., inhibiting or reversing ERM in a patient in need thereof, and/or treating retinal diseases or condition associated with vascular leakage, and/or blocking induction of oxidative stress in RPE cells and/or for inhibiting drusen formation. In certain embodiments, nicotinamide or a related compound may be administered with an additional therapy as pan of a “combination therapy.” For example, additional therapies can include an additional treatment (e.g. a conventional therapy) for the disease, disorder or condition of the retina. Such combination therapy can be sequential therapy wherein the patient is treated first with one therapy and then the other, and so on, or all therapies can be administered simultaneously. In either case, these therapies are said to be coadministered. It is to be understood that “coadministered” does not necessarily mean that the drugs and/or therapies are administered in a combined form (i.e., they may be administered separately or together to the same or different sites at the same or different times).

As used herein, the phrase “pharmaceutically acceptable” refers to molecular entities and compositions that are generally believed to be physiologically tolerable and do not typically produce an allergic or similar untoward reaction, such as gastric upset, dizziness and the like, when administered to a human. As used herein, the term “pharmaceutically acceptable derivative” means any pharmaceutically acceptable salt, solvate or prodrug, e.g., ester, of a compound of the present disclosure, which upon administration to the recipient is capable of providing (directly or indirectly) a compound described herein, or an active metabolite or residue thereof. Such derivatives are recognizable to those skilled in the art, without undue experimentation. Nevertheless, reference is made to the teaching of Burger's Medicinal Chemistry and Drug Discovery, 5th Edition, Vol 1: Principles and Practice. Pharmaceutically acceptable derivatives include salts, solvates, esters, carbamates, and/or phosphate esters.

The term “pharmaceutically acceptable derivative” as used herein means any pharmaceutically acceptable salt, solvate or prodrug, e.g., ester, of a compound of the invention, which upon administration to the recipient is capable of providing (directly or indirectly) a compound of the invention, or an active metabolite or residue thereof. Such derivatives are recognizable to those skilled in the art, without undue experimentation. Nevertheless, reference is made to the teaching of Burger's Medicinal Chemistry and Drug Discovery, 5th Edition, Vol 1: Principles and Practice, which is incorporated herein by reference to the extent of teaching such derivatives. Pharmaceutically acceptable derivatives include salts, solvates, esters, carbamates, and/or phosphate esters. Pharmaceutically acceptable derivatives of nicotinamide and related compounds are encompassed by the treatment methods disclosed herein.

In accordance with the present invention, there may be employed conventional molecular biology, microbiology, recombinant DNA, immunology, cell biology and other related techniques within the skill of the art. See, e.g., Sambrook et al., (2001) Molecular Cloning: A Laboratory Manual. 3rd ed. Cold Spring Harbor Laboratory Press: Cold Spring Harbor, N.Y.; Sambrook et al., (1989) Molecular Cloning: A Laboratory Manual. 2nd ed. Cold Spring Harbor Laboratory Press: Cold Spring Harbor, N.Y.; Ausubel et al., eds. (2005) Current Protocols in Molecular Biology. John Wiley and Sons, Inc.: Hoboken, N.J.; Bonifacino et al., eds. (2005) Current Protocols in Cell Biology. John Wiley and Sons, Inc.: Hoboken, N.J.; Coligan et al., eds. (2005) Current Protocols in Immunology, John Wiley and Sons, Inc.: Hoboken, N.J.; Coico et al., eds. (2005) Current Protocols in Microbiology, John Wiley and Sons, Inc.: Hoboken, N.J.; Coligan et al., eds. is (2005) Current Protocols in Protein Science, John Wiley and Sons, Inc.: Hoboken, N.J.; Enna et al., eds. (2005) Current Protocols in Pharmacology John Wiley and Sons, Inc.: Hoboken, N.J.; Hames et al., eds. (1999) Protein Expression: A Practical Approach. Oxford University Press: Oxford; Freshney (2000) Culture of Animal Cells: A Manual of Basic Technique. 4th ed. Wiley-Liss; among others. The Current Protocols listed above are updated several times every year.

### Nicotinamide and Related Compounds

Nicotinamide is a water soluble B vitamin also known as niacinamide and nicotinic acid amide. In cells, niacin is incorporated into nicotinamide adenine dinucleotide (NAD) and nicotinamide adenine dinucleotide phosphate (NADP), although the pathways for nicotinamide and nicotinic acid are very similar. NAD+ and NADP+ are coenzymes in a wide variety of enzymatic oxidation-reduction reactions. Nicotinamide has a wide range of beneficial effects and is highly safe for human use. Nicotinamide promotes the differentiation of RPE stem cells into RPE cell progeny [see. Blenkinsop et al. Methods Mol Biol. 2013; 945:45-65]. Nicotinamide maintains NAD levels that are depleted by oxidative stress and is thereby necessary for cells to withstand oxidative stress induced injury. For example, nicotinamide reduces damage to rabbit and rat retina caused by oxidative stress [see, Tam D et al. Ann N Y Acad Sci. 2005; 1053:258-68; Sheline et al. Mol Vis. 2010 Dec. 8; 16:2639-52.]. Nicotinamide is an epigenetic gene regulator through inhibition of enzymes, including the histone deacytylases (sirtuin) main member SIRT1 (Maiese et al. Molecules. 2009 Sep. 9; 14(9): 3446-3485). Nicotinamide promotes the differentiation of some stem cell types into various types of progeny, e.g., pluripotent stem cells into RPE progeny or pancreatic progeny. Nicotinamide inhibits the differentiation of some stem cell types, e.g., CD 34 hematopoietic stem cells, induces proliferation of some cell types, e.g., pluripotent stem cells, and inhibits the proliferation of certain other cell types, e.g., melanoma cells.

Nicotinamide also prevents the induction of EMT via inducers of cellular reactive oxygen species (ROS) production such as TNFα. ROS production, as well as the cellular response to ROS and oxidative stress induced by cellular mediators such as TNFα, depletes the cellular NAD pool, and nicotinamide is therefore an inhibitor of the mechanism by which TNFα acts. TNFα and TGFβ are potent inducers of EMT in RPE cells, and nicotinamide antagonizes this effect. Therefore the present disclosure also encompasses the use of antagonists of TNFα for treating the diseases and disorders described herein. Non-limiting examples of TNFα antagonists include, e.g., small molecule inhibitors of TNFα and antibodies such as infliximab (Remicade, Centocor), etanercept (Enbrel, Amgen), Adalimumab (Humira, Abbott), golimumab (Simponi, Centocor) and certolizumab (Cimzia, UCB). Of course, the skilled artisan will appreciate that the present disclosure is not limited to the above-given examples of TNFα antagonists, which are known or will be known in the art. Nicotinamide may also be administered in a combination therapy with a TNFα antagonist, as discussed in more detail below.

As discussed above, while not intending to be bound by any particular theory or mechanism of action, it is presently discovered that nicotinamide stabilizes RPE cells and protects against RPE atrophy and/or change in phenotype, e.g. by maintaining and/or enhancing RPE morphology, by inhibiting RPE cell proliferation, and/or by inhibiting RPE cells from undergoing epithelial to mesenchymal transition (EMT), which can lead to epiretinal membrane (“ERM”) formation and the development of retinal traction. It is to be understood that nicotinamide-related compounds, however, are also encompassed by the methods disclosed herein. In particular, compounds that raise levels of cellular NAD are also encompassed herein, including nicotinic acid, nicotinamide mononucleotide and NAD itself. Further encompassed are compounds that decrease the consumption of NAD by intracellular enzymes, as this also lead to elevation of NAD. Major NAD consuming enzymes include, e.g., PARP, CD38 and sirtuins, and inhibition of these enzymes can enhance NAD levels in cells (Escande et al Diabetes 62:1084-1093, 2013; Liu et al., Neuromol Med (2009) 11:28-42; Surjana Journal of Nucleic Acids Volume 2010, Article ID 157591, 13 pages doi:10.4061/2010/157591).

It is well within the ability of a person of ordinary skill in the art to determine whether a compound raises the cellular level of NAD in, e.g., an RPE cell. In vitro assays may be used to make such determination. Thus, such compounds (i.e., compounds that raise cellular levels of NAD) may be identified by any suitable screening assay known in the art.

### Uses of Nicotinamide and Nicotinamide-related Compounds

Epiretinal membrane formation is a condition characterized by the proliferation of RPE cells, their migration over the inner retina and into the vitreous (often via defects in the retina caused by retinal tears or surgical manipulations). While not intending to be bound by any particular theory or mechanism of action, nicotinamide has been discovered to stabilize the differentiated RPE phenotype and to prevent RPE cells from undergoing epithelial-mesenchymal transition (EMT). RPE cells that undergo EMT become fibroblastic and drive retinal scarring and epiretinal membrane formation leading to retinal traction (Li H, Wang H, Wang F, Gu Q, Xu X (2011) Snail Involves in the Transforming Growth Factor β1-Mediated Epithelial-Mesenchymal Transition of Retinal Pigment Epithelial Cells. PLoS ONE 6(8): e23322). Further, the formation of epiretinal membranes underlies disease states such as macular pucker, proliferative vitreoretinopathy (PVR) or preretinal fibrosis. Moreover, contraction of epiretinal membranes underlies disease states such as vitreomacular traction or tractional retinal detachment. A key part of this process is the conversion of RPE cells to mesenchymal phenotypes (through EMT), such as myofibroblasts. Membranes containing abnormal RPE phenotypes having characteristics of myofibroblasts undergo contraction that results in vitreoretinal traction and retinal detachment. Surgical removal of epiretinal membranes, which is the current standard of care, has significant risk to cause blindness. The presently disclosed methods for inhibiting or reversing ERM thus provide an alternative to surgical removal of epiretinal membranes. Further, a large group of patients are at risk of developing ERM arising from EMT of RPE cells. ERM can occur spontaneously and is associated with, e.g., eye surgery, eye trauma, intraocular inflammation and other causes. Thus, by way of example, a patient undergoing vitreoretinal surgery can receive at the end of the procedure a nicotinamide or a nicotinamide-related compound intraocular formulation (or other suitable formulation and/or route of administration) to achieve levels of the nicotinamide or the nicotinamide-related compound that are sufficient to prevent or reverse epiretinal membrane formation. Similarly, the formulation containing the nicotinamide or nicotinamide-related compound can be injected into the vitreous cavity of patients after ocular trauma or other cause of intraocular inflammation.

Thus, the present disclosure provides methods for inhibiting or reversing ERM in a patient (e.g., a mammal, e.g., a human). The method can include administering to a patient suffering from or at risk of developing ERM an amount of nicotinamide or a nicotinamide-related compound effective for inhibiting proliferation of RPE cells. As discussed above, ERM is an underlying disease state associated with numerous conditions of the retina, including, but not limited to: macular pucker, proliferative vitreoretinopathy, preretinal fibrosis, vitreomacular traction, tractional retinal detachment, and phthsis bulbi. The skilled artisan will understand how to recognize symptoms of and diagnose the above conditions of the retina associated with ERM; however, exemplary signs and symptoms are described below. Also provided herein are methods for inhibiting or reversing EMT in a patient. The methods can include administering to a patient suffering from or at risk of developing EMT an amount of nicotinamide effective for maintaining normal RPE cell morphology.

In some embodiments, the methods disclosed herein include methods of treating macular pucker. Macular pucker occurring spontaneously or after trauma causes distortion of vision known as metamorphopsia, and/or blurring of central vision leading to legal blindness, and/or micropsia where vision is minified in the effected eye, and/or diplopia, a condition in which images from each eye do not fuse but remain separate and distinct. Symptomatic or asymptomatic macular pucker may be observed during ophthalmic examination ophthalmosopically, by OCT or with fluorescein angiography. Macular edema often accompanies macular pucker.

In some embodiments, the methods disclosed herein include methods of treating proliferative vitreoretinopathy. Proliferative vitreoretinopathy causes blurring of central vision, and/or loss of peripheral vision. Extensive epiretinal membrane formation associated with PVR can be observed ophthalmoscopically and/or using ultrasonagraphy.

In some embodiments, the methods disclosed herein include methods of treating preretinal fibrosis. Preretinal fibrosis occurring spontaneously or after trauma causes distortion of vision known as metamorphopsia, and/or blurring of central vision leading to legal blindness, and/or micropsia where vision is minified in the effected eye, and/or diplopia, a condition in which images from each eye do not fuse but remain separate and distinct. Symptomatic or asymptomatic preretinal fibrosis may be observed during ophthalmic examination ophthalmosopically, by OCT or with fluorescein angiography.

In some embodiments, the methods disclosed herein include methods of treating vitreomacular traction. Vitreomacular traction causes blurring of vision, and/or metamorphopsia, and/or micropsia, and/or diplopia. Symptomatic or asymptomatic vitreomacular traction may be observed during ophthalmic examination ophthalmosopically, by OCT or with fluorescein angiography. Macular edema often accompanies vitreomacular traction.

In some embodiments, the methods disclosed herein include methods of treating tractional retinal detachment. Tractional retinal detachment causes a loss of central and/or peripheral vision depending on the location of the epiretinal membrane. These retinal detachments can be observed ophthalmoscopically and/or using ultrasound. Generally, an increase in intraocular inflammation and decrease in intraocular pressure (hypotony) accompany tractional retinal detachment.

In some embodiments, the methods disclosed herein include methods of treating chronic intraocular inflammation. Chronic intraocular inflammation, e.g., after serious injury to the eye, often results in uncontrolled RPE proliferation associated with eye pain, hypotony and shrinkage of the eye known as phthsis bulbi. Symptoms include, e.g., pain, ptosis (eyelid droop), corneal decompensation, hyperemia, and blindness.

The methods of treatment provided herein can prevent or delay the appearance of one or more of the symptoms described above.

Also provided herein are methods for inhibiting the onset of the above-disclosed retinal conditions. The methods include identifying patients at risk of developing ERM, and treating the patients identified as at risk of developing ERM with an amount of nicotinamide or a nicotinamide-related compound effective for inhibiting development of ERM. It is within the skill in the art to determine whether a patient is at risk of developing ERM; however, exemplary signs or symptoms include blurry vision, metamorphopsia, micropsia, the appearance of abnormal cellular material on or within the retina detected ophthalmoscopically or by OCT (optical coherence tomography), leakage of fluorescein dye detected using fluorescein angiography, macular edema, an occlusion of a retinal vein, diabetic retinopathy, retinal vasculitis, retinal hemorrhage, a retinal break or trauma, the presence of vitreous cells, or cells in the anterior chamber, haziness (flare) in the anterior chamber or vitreous, abnormal cells located on, within or under the retina, or abnormal differentiation of RPE cells evidenced by pigment hypertrophy, pigment hypotrophy, or an excess accumulation of cells, on, within or under the retina.

In certain eye conditions, the normal RPE phenotype changes to an abnormal phenotype with weakened or absent tight junctions. Leakage through vascular tight junctions contributes to retinal edema. Several retinal diseases involve leakage (decreased resistance) through the RPE tight junctions due to weakened tight junctions, resulting in retinal or macular edema. Cystoid macular edema (CME) occurs for example in diabetic retinopathy, intraocular inflammation due to uveitis, vasculitis, trauma or surgery. Improving tight junctional resistance with nicotinamide or a nicotinamide-related compound can decrease leakage into the retina to eliminate the cause of retinal edema and treat vision loss associated with retinal edema. Thus, also provided herein are methods for treating retinal diseases or conditions associated with CME, e.g., intraocular inflammation due to uveitis, vasculitis, trauma, surgery, vision loss, and collagen vascular disease (e.g., Behcets disease and sarcoidosis) by administering to a patient in need thereof a nicotinamide or a nicotinamide-related compound. The loss of tight junction integrity can be clinically measured by the leakage of intravascular fluorescein through the RPE layer and the retinal vasculature. Fluorescein leakage observed in exudative AMD, diabetic macular edema, Irvine-Gass type cystoid macular edema and other types of macular edema can also be treated with nicotinamide according to the methods disclosed herein.

In another example, RPE cells are susceptible to oxidative damage leading to cell death and atrophy. Stabilizing the normal RPE phenotype with nicotinamide prevents onset of the pathologic RPE phenotype and subsequent stress-induced RPE cell death. Thus, nicotinamide can be used to treat diseases associated with RPE cell death and RPE atrophy. Accordingly, in one embodiment, provided herein is a method for inhibiting a pathologic condition associated with oxidative damage-induced RPE atrophy in a patient. The method can include administering to a patient in need thereof an amount of nicotinamide or a nicotinamide-related compound effective for inhibiting stress-induced is cell death. Inhibition of cell death can be determined, e.g., by direct observation of a loss of pigmentation within the RPE layer ophthalmoscopically, as hyperfluorescence due to a window defect in the RPE layer using fluorescein angiography or with OCT.

Age-related macular degeneration (AMD) is a condition associated with abnormal RPE cell phenotypes. The formation of drusen deposits between the RPE and Bruchs membrane is associated with early AMD (also known as dry AMD). Abnormal RPE cell phenotypes produce the proteins that constitute drusen. In addition, RPE cells of an abnormal phenotype that over-expresses drusen protein are found overlying drusen in the eyes of patients with dry AMD. Thus, abnormal RPE phenotypes are associated with drusen, the hallmark of early, or dry, AMD. Dry AMD may progress to atrophic AMD with diffuse loss of RPE cells or Geographic Atrophy (GA)), where round areas of RPE cells are lost. The loss or atrophy of RPE cells in atrophic AMD and GA may progress to atrophy of the overlying neural retina and associated loss of vision. About 10% of dry AMD cases progress to wet AMD, when underlying choroidal vessels invade through Bruchs membrane weakened by drusen and/or RPE atrophy. When choroidal vessels invade into the RPE layer, dry AMD is known to have progressed to wet, or exudative, AMD. In dry AMD, the normal barrier to choroidal vessel ingrowth formed by the RPE layer and Bruchs membrane is stressed. Oxidative stress has a key role in maintaining the RPE/Bruchs barrier. Damage to the RPE resulting in cell death and/or change into pathologic phenotypes caused by oxidative stress weakens Bruchs membrane which has an important role in AMD pathogenesis and favors progression to wet AMD.

Thus, also disclosed herein are methods for the treatment of atrophic AMD and GA. While not intending to be limited to any particular theory or mechanism of action, treatment of atrophic AMD and GA patients with nicotinamide or a nicotinamide-related compound is believed to be achieved by preventing the RPE cells from succumbing to oxidative stress, which in turn decreases the rate of RPE cell death and RPE atrophy. See. e.g., Example 6, which demonstrates that nicotinamide reduces the effect of oxidative stress, as measured by up-regulation of TIMP3, in cultured RPE cells. Another utility of nicotinamide or a nicotinamide-related compound, as provided herein, is the treatment of neovascular AMD. It is presently discovered that, in some patients, RPE surround the neovascular lesions associated with neovascular AMD and is associated is with reduced edema, reduced choroidal neovascularization (CNV), and reduced growth of CNV lesions. This wound healing process mediated by RPE cells seals off the neaovascular lesion to reform an intact RPE layer separating the retina from the invading choroidal blood vessels (Miller H, Miller B, Ryan S J. The role of the retinal pigmented epithelium in the involution of subretinal neovascularization. Invest Ophthalmol Vis Sci. 1986; 27:1644-1652). Once the RPE has reformed an intact layer, the neovascular lesion is stabilized. Thus, administration of nicotinamide or a nicotinamide-related compound to these patients can promote the RPE layer at the site of the neovascular lesion and thereby promote wound healing to more effectively seal off the neovascular lesion and reduce the need for anti-neovascular treatment. While not intending to be bound by any particular theory or mechanism of action, it is believed that treatment with nicotinamide or a nicotinamide-related compound can lead to: the production of a larger proportion of RPE cells surviving the inflammatory insult which occurs in the neovascular lesion; enhancement of the normal RPE phenotype which migrates to the site to seal the lesion; stabilization of the RPE phenotype; and inhibition of the differentiation of the RPE into undesirable cell types such as fibroblasts.

In another specific example, in certain eye conditions, the differentiated RPE cell phenotype changes to an abnormal, pathologic phenotype that over-expresses drusen protein (De, et al. Archives Ophthal 2007). Drusen protein overexpression is associated with drusen formation and with dry AMD. Thus, a method for inhibiting this phenotypic change by administering nicotinamide or a nicotinamide-related compound can be used to prevent drusen protein over-expression and drusen and thus to treat dry AMD.

In a particularly common and devastating advanced form of wet AMD, subretinal proliferation of RPE contributes to the formation of complex subretinal tissue accumulation consisting of abnormal RPE phenotypes and mesenchymal cells known as a disciform lesion. Administration of nicotinamide in a patient with advanced neovascular AMD can limit RPE proliferation and thereby limit the growth and size of such disciform lesions. Thus, also provided herein are methods for treating advanced, disciform, wet AMD, wherein the method includes administering to a patient nicotinamide or a nicotinamide-related compound in an amount effective for inhibiting RPE proliferation and EMT.

Also provided herein are in vitro methods for stabilizing the differentiated RPE cell phenotype (characterized, e.g., by cobblestone morphology in cell cultures) and for stabilizing the tight junctions of RPE cells in culture. See, e.g., Examples 2 and 3, below, which demonstrate that nicotinamide enhances the RPE phenotype and increases the epithelial resistance of confluent RPE cultures as determined by the expression of the tight junction marker ZO-1. Non-limiting examples of suitable methods for culturing RPE cells according to the present disclosure are described in Example 1. Further, as demonstrated herein (see, e.g., Example 6, below) exposure of cultured RPE cells to nicotinamide also results in decreasing the cells' sensitivity to various insults, and causes the RPE to maintain their fully differentiated phenotype in the face of stress. This property of nicotinamide and nicotinamide-related compounds is thus useful for the production of RPE cells, e.g., for scientific research or for use in therapeutic applications such as cell transplants. In addition, cells treated with nicotinamide or a nicotinamide-related compound are more resistant to insult and can be shipped from laboratory to laboratory more easily, resulting in higher quality cells on arrival and less time spent acclimating or redifferentiating the cells upon receipt.

The ability of nicotinamide and nicotinamide-related compounds to stabilize the phenotype of RPE cells is also useful in the production of RPE cells or RPE progenitors for therapeutic use. The use of nicotinamide or a nicotinamide-related compound in the culture medium prior to transplantation decreases the probability that the transplanted RPE cells will dedifferentiate or transdifferentiate and thus improves the safety of the preparation. RPE cell transplantation is under active development to replace diseased RPE in conditions such as but not limited to dry AMD, wet AMD, Stargadts disease, and rips of the RPE. Similarly, nicotinamide or a nicotinamide-related compound can be administered into the eye after transplantation to stabilize the phenotype of transplanted RPE cells.

In other embodiments, nicotinamide or a nicotinamide-related compound can be administered to a patient in order to stabilize the RPE phenotype and to terminate pharmacologically induced RPE proliferation (e.g., induced by certain therapies under development to treat diseases of RPE atrophy). Generally, the methods include exposing is cells to (e.g., administering to a patient) an effective concentration of nicotinamide or other nicotinamide-related compound that raises cellular NAD levels (e.g. relative to prior to treatment). The method can further include reducing the nicotinamide concentration and/or frequency of treatment and/or stopping treatment completely once the desired differentiation state of the RPE has been reached.

Any of the above-described treatment methods can be administered in a combination therapy with another agent or therapy. For example, nicotinamide may be administered in a combination therapy with a TNFα antagonist. Further, for example, nicotinamide or a nicotinamide related compound can be administered in a combination therapy with a conventional treatment such as, but not limited to, vitreoretinal surgery. Other conventional treatments include, e.g., retinal laser photocoagulation or cryotherapy, intraocular injection of anti-VEGF antibodies (e.g., avastin, lucentis, eylea, etc.), or intraocular injection of steroid (e.g., triamcinolone, ozurdex, dexamthsone, etc.), or photodynamic laser therapy, or therapies under development such as injection of factors to stimulate RPE proliferation or transplantation of cells to replace damaged RPE. As discussed above, such combination therapy can be sequential therapy wherein the patient is treated first with one therapy and then the other, and so on, or all therapies can be administered simultaneously. In either case, these therapies are said to be coadministered. It is to be understood that “coadministered” does not necessarily mean that the drugs and/or therapies are administered in a combined form (i.e., they may be administered separately or together to the same or different sites at the same or different times).

### Formulations

While it is possible to use a composition provided herein (e.g., a composition containing nicotinamide or a related compound) for therapy as is, it may be preferable to administer it in a pharmaceutical formulation, e.g., in admixture with a suitable pharmaceutical excipient, diluent, or carrier selected with regard to the intended route of administration and standard pharmaceutical practice. Accordingly, in one aspect, the present disclosure provides a pharmaceutical composition or formulation comprising at least one active composition (e.g. nicotinamide or related compound), or a is pharmaceutically acceptable derivative thereof, in association with a pharmaceutically acceptable excipient, diluent, and/or carrier. The excipient, diluent and/or carrier must be “acceptable” in the sense of being compatible with the other ingredients of the formulation and not deleterious to the recipient thereof, as defined above.

The compositions for use in the presently disclosed methods are formulated for administration in any convenient way for use in human or veterinary medicine. In one embodiment, nicotinamide or related compound (i.e., active ingredient) can be delivered in a vesicle, including as a liposome (see Langer, Science, 1990; 249:1527-1533; Treat et al., in Liposomes in the Therapy of Infectious Disease and Cancer, Lopez-Berestein and Fidler (eds.), Liss: New York, pp. 353-365 (1989); Lopez-Berestein, ibid., pp. 317-327; see generally ibid.). In yet another embodiment, the nicotinamide or nicotinamide related compound or TNFα antagonist (i.e., active ingredient), or any combination thereof, can be delivered in a controlled release form. For example one or more compounds of the present disclosure may be administered in a polymer matrix such as poly(lactide-co-glycolide) (PLGA), in a microsphere or liposome implanted subcutaneously, or by another mode of delivery (see Cao et al., 1999, Biomaterials, February; 20(4):329-39). The microspheres of the present invention may also be composed of PLGA and anhydrous poly-vinyl alcohol (PVA). Edlund et al. “Degradable Polymer Microspheres for Controlled Drug Delivery”, Advances in Polymer Science Vol. 157, 2002, 67) lists on page 77 a number of different degradable polymers investigated for controlled drug delivery applications (e.g. polyglycolide, polylactide, etc.). See also U.S. Patent Application Publication No. 2010/0021422 for a detailed discussion of suitable sustained release compositions and Gupta et al. (Nanomedicine. 2010 April; 6(2):324-33) for a discussion of PLGA nanoparticles for sustained ocular drug delivery. Further, sustained release technologies are well known and several are in clinical use in ophthalmology, including, e.g., Retisert™ (fluocinolone acetonide intravitreal implant) 0.59 mg (Bausch, Rochester. NY) and Ozurdex® (dexamethasone intravitreal implant) 7 mg (Allergan Inc., Irvine, Calif.).

In certain embodiments, a compound disclosed herein is formulated for intraocular administration (e.g., eye drop, intravitreal, etc.). Any formulation known in the art suitable for intraocular administration may be used. In some embodiments, the composition is especially adapted for administration into or around the eye (e.g., intraocular administration). For example, a composition can be adapted to be used as eye drops, or injected into the eye, e.g., using peribulbar or intravitreal injection. Such compositions should be sterile and substantially endotoxin-free, and within an acceptable range of pH. Certain preservatives are thought not to be good for the eye, so that in some embodiments a non-preserved formulation is used. Formulation of eye medications is known in the art, see, e.g., Ocular Therapeutics and Drug Delivery: A Multi-Disciplinary Approach, Reddy, Ed. (CRC Press 1995); Kaur and Kanwar, Drug Dev Ind Pharm. 2002 May; 28(5):473-93; Clinical Ocular Pharmacology, Bartlett et al. (Butterworth-Heinemann; 4th edition (Mar. 15, 2001)); Ophthalmic Drug Delivery Systems (Drugs and the Pharmaceutical Sciences: a Series of Textbooks and Monographs), Mitra (Marcel Dekker: 2nd Rev&Ex edition (Mar. 1, 2003); Guang W. Lu; Recent Pat Drug Deliv Formul. 2010 January; 4(1):49-57); and Karthika, K. et al.; Int. J. PharmTech Res. 2010,2(1).

The compounds disclosed herein may also be modified, e.g., to enhance bioavailability. For example, various nutrient transporters, which include peptide, amino acid, folate, and monocarboxylic acid transporters, have been reported to be expressed on the retina and blood-retinal barrier. Nicotinamide and related compounds (e.g., a nicotinamide related compound or a TNFα antagonist) can be formulated as prodrugs which target these transporters to ocular bioavailability.

### Administration and Dosage

Compositions and formulations including nicotinamide or a related compound as described herein can be administered topically (e.g., pre-corneal instillation, e.g. eye drops), orally (e.g., a pill), or parenterally, or by any other suitable methods known in the art. The term “parenteral” includes injection or deposition or sustained release via vehicles or devices (e.g., intravenous, or intravitreal). In a particular embodiment, administration of a composition or formulation for use in the presently disclosed methods is intraocular (e.g., intravitreal injection), or orally.

Exemplary approaches for topical and systemic drug delivery to the posterior segment of the eye are reviewed e.g., in Hughes et al. Adv Drug Deliv Rev. 2005 Dec. 13; 57(14):2010-32; Choonara et al. J Pharm Sci. 2010 May; 99(5):2219-39; Fischer et al. Eur J Ophthalmol. 2011; 21 Suppl 6:S20-6; Li et al. Curr Drug Metab. 2013 August; 14(8):857-862; and Gupta et al. supra.

Administration of a composition or formulation disclosed herein can be once a day, twice a day, or more often. Frequency may be decreased during a treatment maintenance phase of the treatment, e.g., once every second or third day instead of every day or twice a day. The dose and the administration frequency can be adjusted based on the judgment of the treating physician, for example taking into account the clinical signs, pathological signs and clinical and subclinical symptoms of a disease of the conditions treated with the present methods, as well as the patient's clinical history. For example, higher doses or frequency of administration, or a longer duration of treatment may be indicated when a patient is showing symptoms of the disease or disorder (e.g., abnormal RPE morphology, uncontrolled RPE proliferation, leaky tight junctions in the RPE, EMT of RPE cells, etc.).

Length of treatment, i.e., number of days, will be readily determined by a physician treating the subject; however the number of days of treatment may range from about 1 day to about 365 days. The efficacy of treatment can be monitored during the course of treatment to determine whether the treatment has been successful, or whether additional (or modified) treatment is necessary.

It will be appreciated that the amount of an agent (e.g., nicotinamide or related compound) disclosed herein required for use in treatment will vary with the route of administration, the nature of the condition for which treatment is required, and the age, body weight and condition of the patient, and will be ultimately at the discretion of the attendant physician or veterinarian. Compositions will typically contain an effective amount of the active agent(s), alone or in combination. Preliminary doses can be determined according to in vitro and/or animal tests, and the scaling of dosages for human administration can be performed according to art-accepted practices.

Effective amounts for inhibiting RPE cell proliferation can be determined, e.g., in an in vitro cell proliferation assay as described in Example 5. A cell proliferation assay takes into account the number of cell divisions that a cell undergoes over a period of time. Cell proliferation can be monitored by counting the number of cells generated from a known starting population over time. Other assays include immunostaining for proliferation markers such as Ki67 and counting the number of Ki67-positive cells in a given population; if proliferation is decreased by nicotinamide exposure this is indicated by reduced incidence of Ki67-positive cells at that time point versus vehicle control. Other proliferation markers that can be used include uptake of thymidine analogues such as BrdU and EdU which are incorporated during S phase; the incidence of BrdU-positive or EdU-positive cells in a population indicates how many cells are proliferative.

Keeping the above discussion in mind, effective amounts of nicotinamide will typically raise the level of cellular NAD, e.g., by at least about, e.g., 1.5-fold, 2-fold, 3-fold, 4-fold, 5-fold, or more, relative to the level of cellular NAD prior to treatment.

Keeping the above discussion in mind, an exemplary dosage range for nicotinamide for administration to humans can be a dose of about 1 μg to about 100 mg.

Keeping the above discussion in mind, an exemplary dosage range for intravitreal administration of nicotinamide to humans is a dose of about 1 μg to about 100 mg. For example, nicotinamide may be injected into the vitreous at the time of a retinal surgery. Further, the administration of nicotinamide may be repeated via intravitreal injection at intervals after surgery.

In another example, a dosage of nicotinamide for periocular (e.g., retrobulbar or into the peribulbar space) administration is between about 1 μg and 500 mg as a solution or as a suspension).

In another example, nicotinamide can be administered orally. For example, it may be desirable to administer nicotinamide orally during a postsurgical period. Non-limiting examples of oral dosages include, e.g., 0.5 grams to 10 grams.

In another example, nicotinamide is administered in a sustained release composition such as PLGA. Sustained release formulations useful in the methods disclosed herein include, e.g., any that release nicotinamide so that a concentration of about 0.1 uM to about 10 mM is achieved in the eye tissue.

Exemplary dosage ranges of nicotinamide for use in vitro, e.g., for culturing RPE cells, are, e.g., from about 0.1 mM to about 10 mM, from about 1 μM to about 100 mM, from about 10 μM to about 50 mM, from about 100 μM to about 10 mM, or from about 1 mM to about 10 mM.

The following examples are meant to illustrate, not limit, the present disclosure.

### EXAMPLES

### Example 1

### Materials and Methods

The following are the materials and methods for the experiments described in the Examples below.

Human RPE Cell Culture and Proliferation Assay

Primary adult RPE cells were obtained from cadaveric human eyes under IRB-approved protocols, within 36-hours of the time of death. The anterior half of the eye was removed followed by the vitreous, and retina, isolating the posterior eyecup with the RPE/Bruch's membrane/choroid complex intact (as described in Blenkinsop T A, et al. The culture and maintenance of functional retinal pigment epithelial monolayers from adult human eye. 2012; Salero E et al. Adult human RPE can be activated into a multipotent stem cell that produces mesenchymal derivatives. Cell Stem Cell. 2012; 10(1):88-95). The RPE were removed and plated at a density of 100,000 cells/well in RPE medium (as described in Maminishkis A, et al. Confluent monolayers of cultured human fetal retinal pigment epithelium exhibit morphology and physiology of native tissue. Invest Ophthalmol Vis Sci. 2006; 47(8):3612-24) containing 10% fetal bovine serum (FBS). Once the primary (passage zero) cells reached confluence (˜20 days), the FBS concentration was reduced to 5%. Then, following the protocol developed by Salero S et al., 2012 (supra), the RPE were activated to proliferate in RPE medium with 5% FBS. The expanded RPE cells were plated at 30,000 cells per well in a 24-well plate and cultured in DMEM/F12, 5% FBS. Proliferation rates were measured by manual and/or automated counting of cell number as a function of time.

Antibodies and Immunostaining

The following antibodies in Table I were used in the experiments described below.

TABLE 1
Antibodies Used in Examples

| Antibody Name | Species | Clonality | Source |
|---|---|---|---|
| ZO-1 | Rabbit IgG | Polyclonal | Invitrogen: 617300 |
| Ki67 | Rabbit IgG | Monoclonal | Thermo: RM-9106-S0 |
| SNAIL | Goat IgG | Polyclonal | R&D Systems: AF3639 |

Cells were fixed with 4% paraformaldehyde for 15 minutes, permeabilized with 0.2% Triton in PBS, blocked with 1% BSA for 30 minutes. Primary antibodies were used at 1:100 dilution in PBS. The corresponding Alexafluor 647 secondary antibodies were used for the respective primary antibody and imaged on a Zeiss Observer.D1 fluorescent microscope.

Gene Expression Analysis

Gene expression experiments were measured by real time PCR (RT-PCR) using specific oligonucleotide primers (synthesized by Integrated DNA Technologies). RNA was isolated from adult human RPE cells and subject to reverse transcription to produce cDNA. The isolated RNA was digested and the first strand cDNA was then used for RT-PCR. The primers were mixed with the cDNA and then subject to repeated amplification cycles in the presence of a fluorescent reporter. Fluorescent reporter was SyberGreen from Applied Biosciences. The amount of fluorescence emitted during the reaction was monitored in real time, and the expression levels of the genes indicated in Table II, below, were quantitated. Fluorescent output was analyzed to determine the cycle threshold or CT value. The gene expression for the product was compared to an internal control (GAPDH or cyclophilin). Table II, below, provides the primer sequences used to detect expression of the listed cell markers.

TABLE II
List of primers used for Real Time PCR on adult human RPE

| | | | Product | | |
|---|---|---|---|---|---|
| | | | Size | Tₐₙₙ | Gene Bank |
| Human Gene | Forward 5′-3′ | Reverse 3′-5′ | (bp) | (° C.) | Accession |
| microphthalmia- | TTGTCCATCTGCCTCT | CCTATGTATGACCAG | 87 | 55 | NM_198178 |
| associated | GAGTAG (SEQ ID NO.: | GTTGCTTG (SEQ ID | | | |
| transcription | 1) | NO.: 2) | | | |
| factor (MITF) | | | | | |
| snail family zinc | TGTCAGATGAGGACA | CTGAAGTAGAGGAGA | 611 | 53 | NM_005985 |
| finger 1 (SNAIL) | GTGGGAAAGG (SEQ ID | AGGACGAAGG (SEQ | | | |
| | NO.: 3) | ID NO.: 4) | | | |
| snail family zinc | AGCGAACTGGACACA | TCTAGACTGGGCATC | 410 | 55 | NM_003068 |
| finger 2 (SLUG) | CATAC (SEQ ID NO.: 5) | GCAG (SEQ ID NO.: 6) | | | |
| twist basic helix- | GTCCGCAGTCTTAGCA | GCTTGAGGGTCTGAA | 156 | 60 | NM_000474 |
| loop-helix | GGAG (SEQ ID NO.: 7) | TCTTGCT (SEQ ID | | | |
| transcription | | NO.: 8) | | | |
| factor 1 (TWIST) | | | | | |
| TIMP | CATGTGCAGTACATCC | CATCATAGACGCGAC | 100 | 55 | NM_000362 |
| metallopeptidase | ATACGG (SEQ ID NO.: 9) | CTGTCA (SEQ ID | | | |
| inhibitor 3 | | NO.: 10) | | | |
| (TIMP3) | | | | | |
| RPE65 | TGGTGTAGTTCTGAGT | AGTCCATGAAAGGTG | 137 | 60 | NM_000329 |
| | GTGGTGGT (SEQ ID | ACAGGGATGTT (SEQ | | | |
| | NO.: 11) | ID NO.: 12) | | | |
| GAPDH | CCCCTTCATTGACCTC | TTGCTGATGATCTTG | 342 | 56 | NM_002046 |
| | AACTACA (SEQ ID | AGGCTGT (SEQ ID | | | |
| | NO.: 13) | NO.: 14) | | | |
| Cyclophilin | CTTGTCAATGGCCAAC | GCCCATCTAAATGAG | 82 | 60 | NM_004792 |
| | AGAGG (SEQ ID NO.: 15) | GAGTTGCGT(SEQ ID | | | |
| | | NO.: 16) | | | |

Microscopy

Cultures were imaged at 32× on a Zeiss Observer.D1 fluorescent microscope.

Measurement of Transepithelial Resistance

The transepithelial resistance (TER) is measured during and after treatment of hRPE cells cultured in 12-well 0.4 μm transwell inserts using the EVOM2 World Precision Instruments Volt Ohm Meter protocol. Resistance is measured in Ω-cm² and recorded prior to treatment to confirm that all wells are at approximately the same TER. The TER of a cell-free transwell insert of ˜110 Ω-cm² is subtracted to obtain the resistance of the cellular epithelium. TER is measured in each well after treatment to assess the change caused by nicotinamide.

Oxidative Stress Analysis

TBHP (Sigma-Aldrich) was utilized as an inducer of oxidative stress following prior studies of the RPE stress response (Glotin A L, et al. Prematurely senescent ARPE-19 cells display features of age-related macular degeneration. Free Radic Biol Med. 2008; 44(7):1348-61; Weigel A L, et al. Microarray analysis of H2O2-, HNE-, or tBH-treated ARPE-19 cells. Free Radic Biol Med. 2002; 33(10): 1419-32)). For each experiment, confluent human RPE (hRPE) cells in three wells of a 12-well transwell insert or a 24-well plate were fed with minimal RPE medium containing 500 μM TBHP or vehicle for two hours per day for 1-5 days. Following each TBHP exposure, the hRPE were washed twice with Hank's Buffered Salt Solution (Gibco) and then rested in minimal RPE medium for 22 hours. This protocol applied to confluent hRPE cultures with varied concentrations of nicotinamide in the minimal RPE media was used to obtain data describing the effect of nicotinamide on the chronic stress response of hRPE.

Epithelial to Mesenchymal Transition Assay

Epithelial to mesenchymal transition (EMT) was determined in fibroblastic cultures as follows: cells were plated 30,000 cells per 1 cm² tissue culture plate in DMEM/F12 with 5% FBS with the addition of 10 ng/ml TGFβ and 10 ng/ml TNFα to induce epithelial to mesenchymal transition (EMT). The extent of EMT was determined by the morphologic changes and immunohistochemical stains described above.

### Example 2

### Effect of Nicotinamide Treatment on RPE Morphology In Vitro

This example demonstrates that nicotinamide treatment preserved RPE phenotype, including characteristic cobblestone morphology and maintenance of tight junctions.

Cultured RPE cells were analyzed by light microscopy. RPE cells cultured in the absence of nicotinamide lost their characteristic cobblestone morphology, were biased towards a fibroblastic appearance, lost normal cell-cell junctions, and exhibited reduced staining for the junctional marker ZO1 (FIG. 1A). However, RPE cells cultured in the presence of 10 mM nicotinamide maintained their characteristic cobblestone morphology, were polarized, and had high levels of ZO1 staining. The RPE cells cultured in the presence of nicotinamide also exhibited other features of polarization characteristic of native RPE cells, including apical microvilli, tight junctions and a basally located nucleus.

### Example 3

### Effect of Nicotinamide Treatment on RPE Transepithelial Resistance In Vitro

This example demonstrates that nicotinamide treatment increases transepithelial resistance and improves tight junction protein in adult human RPE.

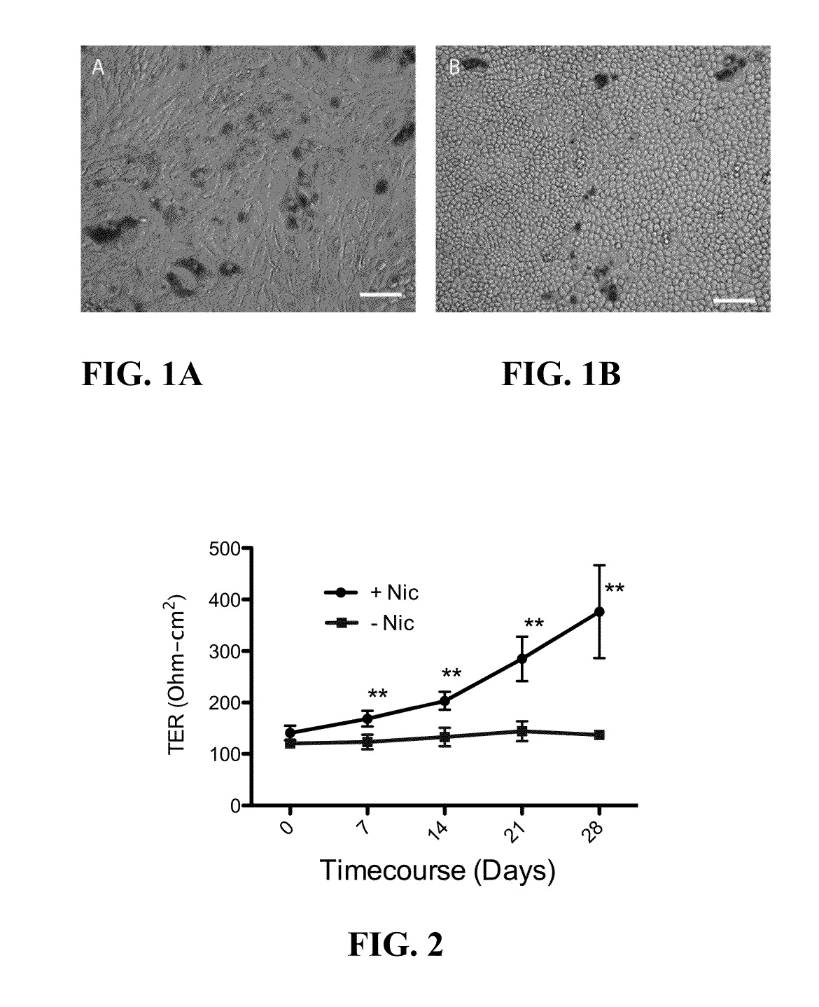

Adult human RPE were plated on semi-permeable polyester transwells and transepithelial resistance was measured weekly for one month. In the presence of nicotinamide, transepithelial resistance increased over time (FIG. 2).

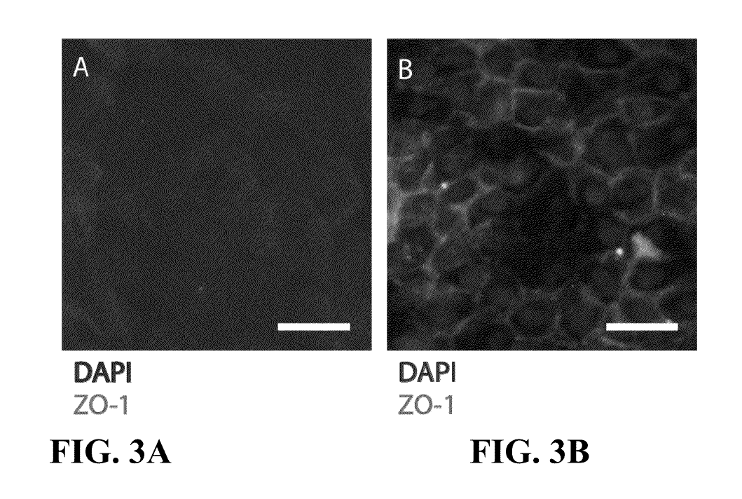

Further, culture of human RPE cells in the presence of 10 mM nicotinamide resulted in localized expression of tight junction protein ZO-1 peripherally around the cell borders (FIG. 3B), whereas this pattern of ZO-1 expression was not observed in control RPE cells (no nicotinamide) (FIG. 3A).

### Example 4

### Effect of Nicotinamide on RPE Identity

This example demonstrates that nicotinamide preserves and enhances RPE identity, and prevents epithelial to mesenchymal transition.

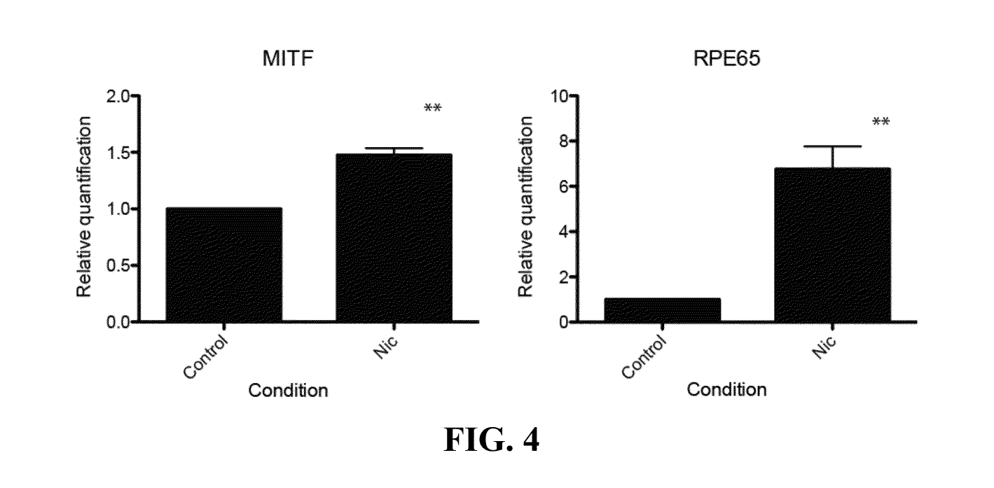

The mRNA expression levels of RPE markers MITF and RPE65, and mesenchymal markers SNAIL, SLUG and TWIST was determined in RPE cells treated with or without nicotinamide using real-time PCR. Gene expression analysis demonstrated that the expression of RPE unique RNA transcripts increased in the presence of 10 mM nicotinamide (FIG. 4). Further, expression of transcription factors SNAIL, SLUG and TWIST, which are involved in the epithelial to mesenchymal transition, decreased in the presence of 10 mM nicotinamide.

A dose response curve for the effect of nicotinamide on the prevention of epithelial to mesenchymal transition was determined using SNAIL expression as a read-out. RPE cells were cultured at 30,000 cells/well in a 24 well plate and cultured for 5 days in the presence of media containing 10 μM, 100 μM, 1 mM or 10 mM nicotinamide or control (no nicotinamide). The media was changed 3 times, and replaced with media containing the same concentration of nicotinamide, after which the cells were fixed and stained with fluorescently-labeled anti-SNAIL antibody. At all concentrations, SNAIL staining was decreased by application of nicotinamide.

### Example 5

### Effect of Nicotinamide on RPE Cell Proliferation

This example demonstrates that nicotinamide decreases RPE cell proliferation.

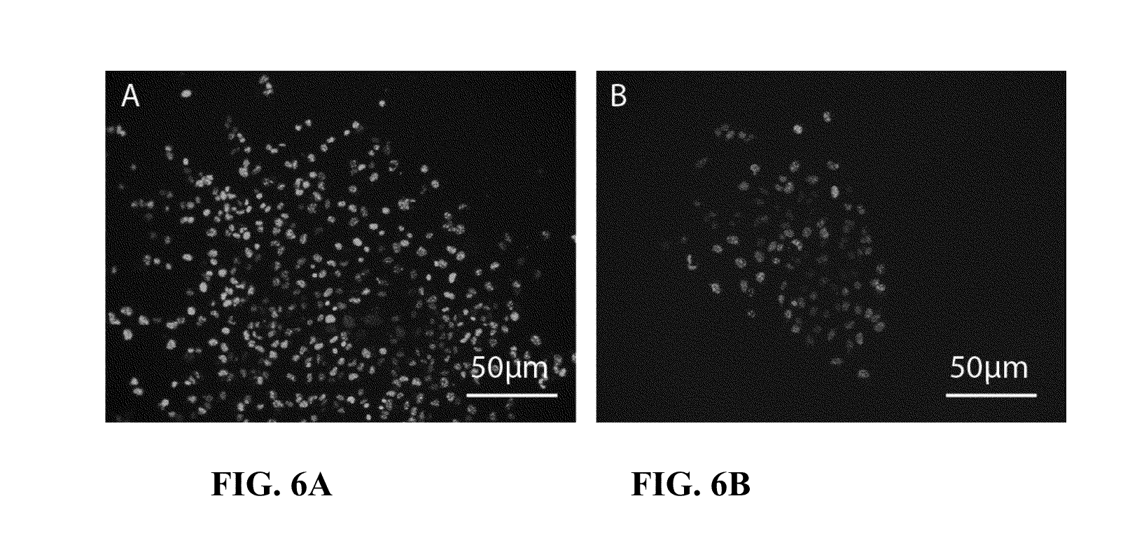

RPE cells were plated at 30,000 cells per well in a 24 well plate and cultured for 5 days in DMEM/F12, 5% FBS and cell proliferation was measured. The RPE cells proliferated extensively within 5 days in the absence of nicotinamide (FIG. 6A), whereas RPE that were cultured in the presence of 10 mM nicotinamide maintained a lower level of proliferation, as indicated by decreased immunostaining for the cell proliferation marker Ki67, and by a decrease in the total cell number (FIG. 6B).

### Example 6

### Effect of Nicotinamide on Induction of Oxidative Stress in RPE Cells

This example demonstrates that nicotinamide blocks oxidative stress induced in RPE cells following exposure to tert-butyl hydroperoxide (TBHP).

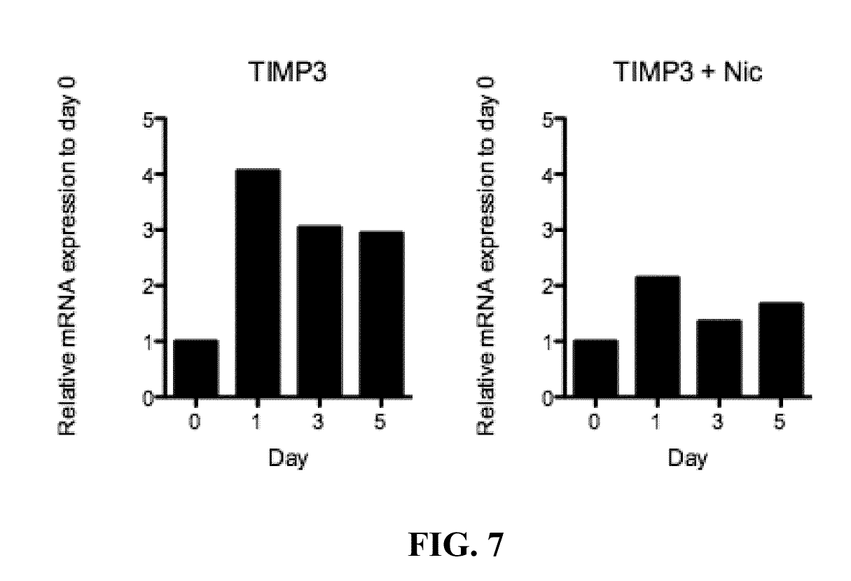

Adult human RPE were cultured for 2 months until they formed an epithelial monolayer, then treated daily for 2 hours with 500 μM TBHP in the presence or absence of nicotinamide. TIMP-3 gene expression was assayed by quantitative PCR, and the relative expression of TIMP-3, relative to day 0, was determined. In the presence of nicotinamide, TBHP-induced TIMP3-expression was suppressed (FIG. 7).

### Example 7

### RPE Thickening Around Choroidal Neovascularization in AMD

This example presents data showing that a ring of pigmentation forms around choroidal neovascularization (CNV) lesions in a subset of patients with age-related macular degeneration (AMD), and is associated with reduced edema and CNV growth, indicating a latent RPE wound healing mechanism in human wet AMD. These observations show that RPE activation, and incomplete RPE-mediated repair, occurs in patients with exudative, e.g., wet, AMD.

Changes in subretinal pigmentation are common in AMD. Areas of sub-retinal pigment hypertrophy and hypotrophy have been described in non-exudative (dry) AMD and exudative (wet) AMD (reviewed in Bonilha, Age and disease-related structural changes in the retinal pigment epithelium. Clin Ophthalmol. 2008 June; 2(2): 413-424). RPE cell hyperplasia along with increased pigment within RPE cells have been reported to contribute to this hyperpigmentation (see, Lopez P F et al. Retinal pigment epithelial wound healing in vivo. Arch Ophthalmol 1995; 113:1437-46; Bonhila, 2008). RPE hyperplasia occurs in association with drusen (see, Bressler N M et al. Clinicopathologic correlation of drusen and retinal pigment epithelial abnormalities in age-related macular degeneration. Retina 1994; 14:130-42) and CNV (see, Miller H et al. The role of the retinal pigmented epithelium in the involution of subretinal neovascularization. Invest Ophthalmol Vis Sci. 1986; 27:1644-1652), and as a prominent feature of disciform scars in advanced AMD (Sarks, S. Ageing and degeneration in the macular region: a clinico-pathological study. Br J Ophthalmol. 1976 May; 60(5): 324-341), which, in extreme cases, can simulate a tumor (see, Eyal Margalit and Neil M. Bressler (2003) Retinal Pigment Epithelium Tumorlike Lesion Arising From an Area Treated With Laser PhotocoagulationArch Ophthalmol. 121(1):130). Wound healing by RPE proliferation that occurs after RPE rips in animal models (see, Wilson J. Heriot, Robert Machemer. Pigment epithelial repair. Graefe's Archive for Clinical and Experimental Ophthalmology January 1992, Volume 230, Issue 1, pp 91-100) also occurs clinically in patients (see, Peiretti et al. Repopulation of the retinal pigment epithelium after pigment epithelial rip. Retina 2006; 26 (9) 1097-1099) and after laser damage to the RPE in animal models of wet AMD (see, Pollack A, Korte G E. Repair of retinal pigment epithelium and its relationship with capillary endothelium after krypton laser photocoagulation. Invest Ophthalmol Vis Sci. 1990; 31:890-898).

Materials and Methods:

Permissions to use images for research purposes were obtained from 33 patients with pigment adjacent to a CNV lesion, from more than 400 AMD patients undergoing fundus photography. Prominent increases in pigment in the absence of significant hemorrhage or fibrous proliferation was observed in 6 patients, and three of these were selected on the basis of having the most complete data illustrating the formation of a pigment ring around CNV. Images were collected using a Zeiss FF450 or Topcon 50× fundus camera with MRP imaging software (Escalon) and a Zeiss 3, Optko or Heidleberg optical coherence tomography (OCT) (see Jaffe G J, Caprioli J. Optical coherence tomography to detect and manage retinal disease and glaucoma. Am J Ophthalmol. 2004 January; 137(1): 56-69).

In all patients, anti-VEGF was initiated on presentation of wet AMD. Intraocular injections per standard protocol were delivered monthly initially and then the treatment interval was extended depending on recurring symptoms and macular edema. In cases of a complete pigment surround, treatment intervals were extended from several months to a maximum of 2 years. Patients received avastin, lucentis or eylea using standard treatment protocols except when treatment intervals were increased in the presence of pigment capping (for standard treatment protocols, see, Zampros et al. Antivascular endothelial growth factor agents for neovascular age-related macular degeneration. J Ophthalmol. 2012; 2012).

Results:

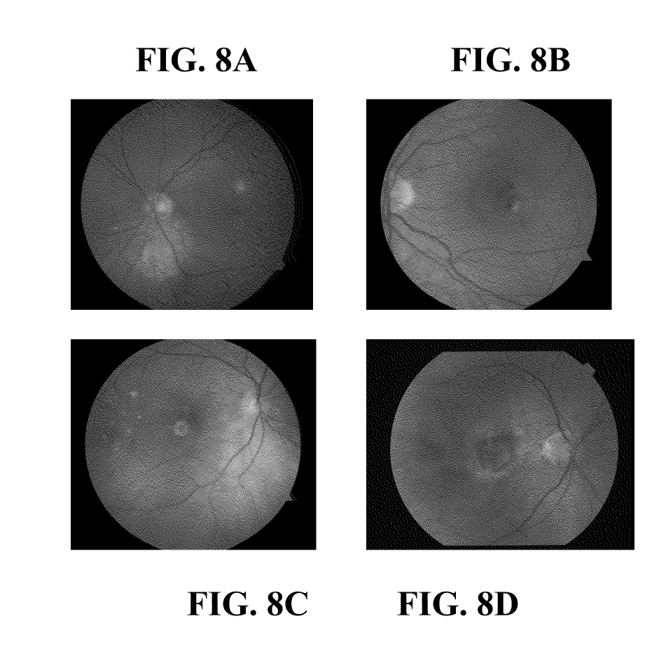

Pigment around CNV lesions varied from partial to complete surrounding of a lesion, as illustrated by the pigment rings in FIGS. 8A-D and 9A-9D. FIG. 8A shows dense brown pigment surrounding about 270 degrees of a lesion while FIGS. 8B-8D show complete, 360 degree pigment capping. The pigment rings shown developed over several weeks after anti-VEGF treatment was initiated.

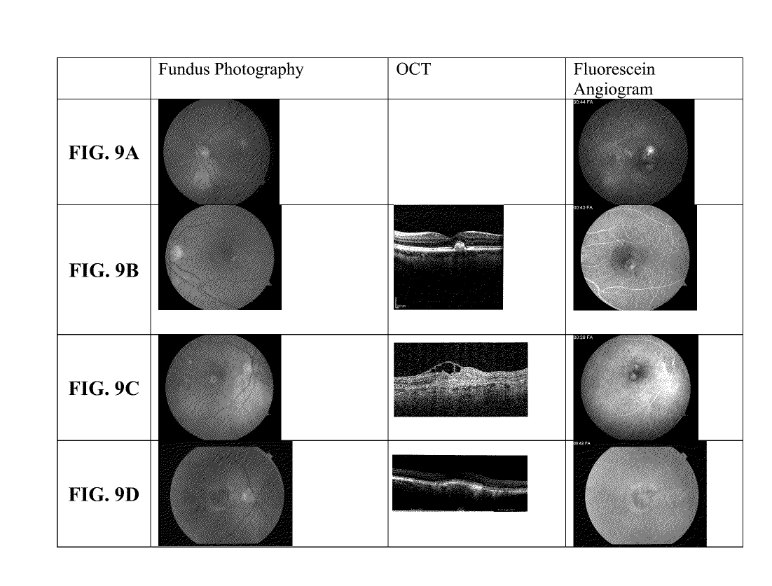

Pigment hypertrophy was accompanied by increased thickening on OCT at the level of the RPE (FIGS. 9B-D). Retinal edema decreased adjacent to pigment hypertrophy and RPE thickening (FIGS. 9 B-D). Diffuse fluorescence leakage was reduced adjacent to pigment compared to areas of CNV not associated with pigment hypertrophy (FIG. 9B). OCT showed edema corresponding to leakage in the direction of pigment (FIG. 9D). In each case, thickening at the level of the RPE was observed on OCT (FIGS. 9B-D).

These results suggest that CNV elicits a proliferative response of the RPE to reform the barrier against choroidal vessels protecting overlaying neural retina, and raise the possibility that activation of a resident RPE progenitor population may slow progression of exudative (wet) AMD.

### Example 8

### Nicotinamide Reverses Epithelial to Mesenchymal Transition

This example demonstrates that nicotinamide treatment reverses epithelial to mesenchymal transition (EMT) in fibroblastic cultures derived from RPE.

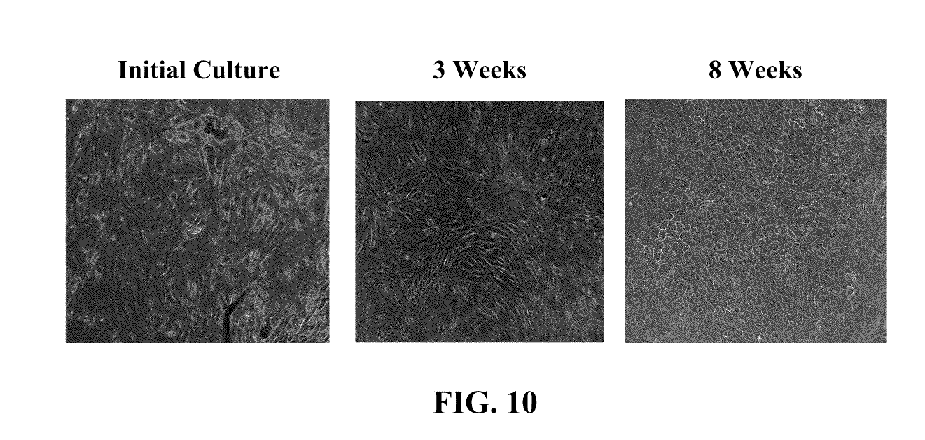

Epithelial cells were plated at 30,000 cells/cm² and cultured until confluent (left image), after which 10 mM nicotinamide was added to the media, which was changed 3 times a week for 8 weeks (FIG. 10). The swirling fibroblastic morphology seen prior to addition of nicotinamide seen at the ‘3 week’ stage indicate the RPE cells have undergone EMT, losing their cobblestone morphology. The micrograph at ‘8 weeks’ shows that after application of nicotinamide for 5 weeks, the RPE cells revert to their physiologic cobblestone morphology. In summary, FIG. 10 shows that nicotinamide reverses EMT in RPE cells that have previously undergone EMT.

### Prophetic Example 1

### Nicotinamide Reverses Epithelial to Mesenchymal Transition

AMD patients are treated with nicotinamide to repair the RPE layer. For example, changes in the RPE layer occur as AMD progresses, including pigment hypertrophy and hypotrophy within the RPE layer, in which the RPE cell phenotype is altered due to EMT. It was demonstrated above that application of nicotinamide stabilizes RPE cells by preventing EMT. The use of nicotinamide to treat RPE in AMD is expected to stabilize the RPE phenotype and thereby stabilize or reverse the pathologic changes of AMD.

A clinical study of AMD patients treated with nicotinamide compared with the natural course of untreated AMD is needed for FDA evaluation of this proposed use. As described above, an intraocular formulation of nicotinamide is given to AMD patients who are undergoing active pathologic change to the RPE as a therapy to stabilize the RPE and to thereby slow progression of AMD. Similarly, and in addition to the use for AMD described above. RPE undergo EMT in epiretinal membrane formation, and intraocular nicotinamide can be administered to patients in clinical studies to inhibit EMT and thereby treat ERM formation, which causes, e.g., macular pucker, proliferative vitreoretinopathy, preretinal fibrosis, vitreomacular traction, tractional retinal detachment, and phthsis bulbi. Furthermore, EMT of the RPE contributes to loss of the physiologic barrier that RPE constitutes against leakage or choroidal neovascularization. The use of nicotinamide to prevent EMT is expected to prevent breakdown of the RPE barrier, which in turn is useful for treatment of macular edema or choroidal neovascular invasion through the RPE layer, as occurs in inflammatory diseases of the retina or in exudative AMD.

A number of embodiments of the present invention have been described. Nevertheless, it will be understood that various modifications may be made without departing from the spirit and scope of the invention. Accordingly, other embodiments are within the scope of the following claims.

What is claimed is:

1. A method for inhibiting or reversing epiretinal membrane (ERM) formation in a patient, the method comprising administering to a patient suffering from or at risk of developing ERM an amount of nicotinamide effective for inhibiting proliferation of retinal pigmented epithelial (RPE) cells.

2. The method of claim 1, wherein the ERM is the underlying disease state of a condition selected from the group consisting of: macular pucker, proliferative vitreoretinopathy, preretinal fibrosis, vitreomacular traction, tractional retinal detachment, and phthsis bulbi.

3. A method for inhibiting or reversing epithelial mesenchymal transition (EMT) in a patient, the method comprising administering to a patient suffering from or at risk of developing EMT an amount of nicotinamide effective for maintaining or inducing normal RPE cell morphology.

4. A method for treating a retinal disease or condition associated with cystoid macular edema (CME) in a patient, the method comprising administering to a patient in need thereof an amount of nicotinamide effective for reducing leakage through tight junctions.

5. The method of claim 4, wherein the retinal disease or condition is selected from the group consisting oft intraocular inflammation due to uveitis, vasculitis, trauma, surgery, and collagen vascular disease.

6. The method of claim 5, wherein the collagen vascular disease is Behcets disease or sarcoidosis.

7. A method for inhibiting a pathologic condition associated with oxidative damage-induced RPE atrophy in a patient, the method comprising administering to a patient in need thereof an amount of nicotinamide effective for inhibiting stress-induced RPE drusen formation and/or stress-induced RPE cell death and/or RPE cell atrophy.

8. The method of claim 7, wherein the pathologic condition is dry (non-exudative) AMD.

9. A method for inhibiting drusen in a patient, the method comprising administering to a patient in need thereof an amount of nicotinamide effective for stabilizing the RPE phenotype, thereby preventing overproduction of drusen protein and associated drusen.

10. The method of claim 8, wherein the patient is suffering from dry (non-exudative) AMD.

11. A method for treating a patient suffering from or at risk of developing neovascular (wet or exudative) AMD, the method comprising administering to a patient in need thereof an amount of nicotinamide effective for stabilizing the RPE cell barrier to choroidal neovascular ingrowth.

12. The method of claim 1, wherein the patient is a mammal.

13. The method of claim 3, wherein the patient is a mammal.

14. The method of claim 4, wherein the patient is a mammal.

15. The method of claim 7, wherein the patient is a mammal.

16. The method of claim 9, wherein the patient is a mammal.

17. The method of claim 12, wherein the mammal is a human.

18. The method of claim 13, wherein the mammal is human.

19. The method of claim 14, wherein the mammal is human.

20. The method of claim 15, wherein the mammal is human.

21. The method of claim 20, wherein the mammal is human.
